(19) United States
(12) Patent Application Publication
(10) Pub. No.: US 2017/0209215 A1
(43) Pub. Date: Jul. 27, 2017

(54) Method and device for biological tissue regeneration (embodiments)

(71) Applicants: Uladzimir Valiantinavich Khomchanka, Minsk (BY); Dmitry Vladislavovich GORBACH, Minsk (BY); Aliaksandr Valerjavich SUKHADOLAU, Minsk (BY)

(72) Inventors: Uladzimir Valiantinavich Khomchanka, Minsk (BY); Dmitry Vladislavovich GORBACH, Minsk (BY); Aliaksandr Valerjavich SUKHADOLAU, Minsk (BY)

(21) Appl. No.: 15/470,865

(22) Filed: Mar. 27, 2017

Related U.S. Application Data
(63) Continuation-in-part of application No. 14/400,338, filed on Nov. 19, 2014, now abandoned.

(51) Int. Cl. A61B 18/20; A61N 7/00; A61H 23/02

(52) U.S. Cl. CPC A61B 18/203; A61H 23/0245; A61N 7/00; A61H 2201/10; A61N 2007/0034; A61B 2018/00452

(57) ABSTRACT

The proposed method is for the regeneration of biological tissues with restoration of functional properties, characteristics and structure thereof, in which tissues are subjected to a predetermined degree of mechanically-induced trauma through the creation, in the desired areas, of at least one region of interference between acoustic waves generated by at least two sources and propagating in the tissues to be regenerated, with the possibility of subsequent natural regeneration of the corresponding biological tissues in said areas. Also proposed are various embodiments of the above method. To achieve the rejuvenation effect of different biological tissues located at different depths, the microtrauma areas are created with no thermal effect, i.e. neither evaporation nor coagulation of all overlying tissues, i.e. regeneration of the tissues occurs with no fibrous cell growth, suggesting that not only visual but also actual rejuvenation took place.

### CROSS-REFERENCE TO RELATED APPLICATION

This application is a continuation-in-part application of application Ser. No. 14/400,338 filed on Nov. 19, 2014, which is currently pending. The earliest priority date claimed is Nov. 5, 2012.

### FEDERALLY SPONSORED RESEARCH

Not Applicable

### SEQUENCE LISTING OR PROGRAM

Not Applicable

### BACKGROUND

The invention relates to medicine and can be used in surgery, including cosmetic surgery, for example, for treating trophic and slowly healing ulcers, bed sores, burns, scars, etc., as well as for tissue rejuvenation, including skin, in different locations. The method is based on the transmission of non-mechanical energy, in particular, acoustic wave energy, into the human tissue. The invention also relates to different embodiments of the device generating the wave energy necessary for implementing said method.

Modern surgery, including cosmetic surgery, widely uses laser energy, ultrasound energy and similar non-mechanical types of energy for treatment and rejuvenation. Thus there is a known skin rejuvenation method comprising ablation or vaporization of superficial skin layers with carbon dioxide laser radiation at 10.6 um wavelength or with Er laser (EnYAG) at 2.94 gm wavelength (Palomar 2940 Fractional Laser, Deka Smart-Xide DOT, Candela CO2RE) [1]. The therapeutic effect in this case is based on the vaporization of superficial skin layers with minor thermal damage of deep dermic layers, which does not fully destruct all skin layers but stimulates new cell growth. Additional disadvantages of said method are the high trauma rate, long recovery, and wound formation, which carries a high risk of infection, pain, both during the procedure and during recovery, the risk of altered skin pigmentation and scar formation. In addition, the method cannot be used on moving body parts, such as neck, eyelids, etc., since wound healing requires immobilization of the treatment zone.

A method for non-invasive photorejuvenation, wherein radiation penetrates deeper into the skin, causing trauma to collagen fibers and then stimulating new collagen synthesis, is known in the art (Palomar 1540 Fractional Laser, Candela GentleMAX, Candela Smoothbeam) [2]. Said method can be used in the treatment of essentially all skin areas. The disadvantages of said method include inefficient therapeutic and aesthetic effects. The amount of synthesized collagen is insufficient for producing the rejuvenation effect expressed as diminished wrinkle size. The method improves skin color by increasing capillary blood supply and can cause temporary cutaneous edema, which creates a temporary wrinkle-reducing effect.

Methods for non-invasive ultrasonic skin rejuvenation are also known in the art Said methods are used for the treatment of various skin areas in the desired treatment sites. The disadvantages of said methods include the ultrasonic wave front effect on the tissue, which not only impacts the desired site but also the surrounding tissue, which, in turn, enlarges the trauma area and delays recovery.

The method most closely related to the claimed method is the microablative skin photorejuvination [6]. In said method, rather than treating the skin surface with one wide laser beam, the skin surface is treated with a plurality of microbeams.

Each microbeam triggers either coagulation alone, coagulation combined with evaporation, or cutaneous microdomain ablation, depending on spectral and temporal parameters of the applied radiation. Microbeam diameters can span from 1 um to hundreds micrometers, and they can be situated hundreds of micrometers apart. The therapeutic effect of the method is based on the assumption that the removed or damaged tissue will be replaced with new skin cells, and old skin will be completely replaced with new skin in the treatment area over the course of several sessions. Skin microcoagulation with Erbium glass lasers (laser apparatus Fraxel, wavelength 1.54 um) induces thermal destruction of skin cells without evaporation thereof. Radiation causing both skin cell evaporation and coagulation (such as carbon dioxide radiation, wavelength 10.64 um) produces microchannels of the evaporated skin surrounded by a coagulation zone. Erbium radiation laser (wavelength 2.94 pm) causes tissue evaporation as microchannels without coagulation of the surrounding tissue.

Disadvantages of the method are include:

The depth of microtrauma, wherein the rejuvenation effect occurs, is limited by the coagulation or ablation depth, which does not allow for rejuvenation in the deep dermis or hypodermis;

The small depth of microtrauma is not useful for the inhanced tissue regeneration when treating trophic or septic wounds, etc., i.e. when tissue regeneration must occur at a much deeper level;

The method is invasive, which increases the risk of infection in the treated surface;

When tissue is removed, the living tissue is exposed to the environment, which can promote the growth of fibrous tissue instead of full-fledged rejuvenation of the unaltered tissue;

Microablative procedures are painful and thus require the use of anesthetics.

Thus, the object of the present invention is to provide a noninvasive method for the rejuvenation of biological tissue and restoration of functional properties, characteristics, and structure thereof by creating nonthermal microtrauma sites in the desired areas of biological tissue, causing subsequent natural regeneration of the corresponding biological tissue in the desired areas. Nonthermal character of microtraumatising preclude possibility of formed defect replacement by fibrous tissue, stimulate only natural regeneration of biological tissue and restoration of functional properties, characteristics, and structure. Microtrauma (microdestruction) inside the biological tissue should not lead to the formation of microchannels exposed to the aggressive environment, which would completely preclude the formation of fibrous tissue. The treatment should promote the formation of microtrauma sites and later regeneration of both the superficial and deep biological tissue of any localization and any type. The method should also reduce pain and the risk of infection as compared to other methods known in the art, including microablattive methods.

### SUMMARY

The stated objective is achieved in the claimed method for the rejuvenation of biological tissue and restoration of functional properties, characteristics, and structure thereof by creating microtrauma sites in the desired areas of biological tissue, followed by natural regeneration of the corresponding biological tissue in the desired areas with specified mechanically-induced trauma by creating in the desired areas at least one site of acoustic interference generated by at least two sources and propagating in the tissue to be regenerated.

In the claimed method, the powerful common-mode acoustic waves with the desired (calculated) characteristics, such as power, are generated, in general, on the surface to be rejuvenated or the surface of overlying biological tissue. In particular, altering the power of acoustic waves can have a corresponding effect on the predetermined depth of microtrauma areas. The interference effect of interacting waves in the method of the present invention decreases the size and determines the exact location in all directions of the tissue area subjected to microtrauma, which results in a considerably higher efficiency of the directional energy effect and shorter recovery time.

In the claimed method, the tissue trauma is nonthermal and thus, the effect thereof on the biological tissue does not cause evaporation or coagulation. Additionally, since acoustic waves can penetrate the biological tissue at a set depth (defined by the acoustic wave characteristics) with no channel formation, the effect of the present invention does not increase the contact between the living tissue and oxygen, which impedes the growth of fibrous tissue and promotes real and not only visual regeneration/rejuvenation of biological tissue. Energy of the acoustic waves, which is stronger in the desired interference zones, does not destruct tissue but causes trauma to the selected tissue areas. Because regeneration of biological tissue can occur not only as a result of complete tissue cell destruction, but also as a result of partial trauma thereof, the biological tissue in the selected locations are regenerated. Since no complete destruction of deeply underlying cells of biological tissue takes place, recovery time is greatly reduced. Since in the method of the present invention, the trauma area is reduced, the level of trauma can be preset, and the thermal effect is absent, the pain during the procedure is considerably reduced.

The areas of the common-mode acoustic wave sources are preferably 10 nm² to 0.2 mm². Preferably, all epicenters of acoustic waves are located at equal distances from one another, selected from the 10 1.1 m to 1 cm range.

According to the present invention, mechanical trauma zones are preferably formed below the surface of tissue exposed to the environment, without increasing the contact surface of living tissue with the aggressive media. Thus, the claimed method creates microtrauma areas with no expansion of contact areas between the living tissue and the environment, which considerably reduces the risk of infection during recovery in comparison to the methods known in the art.

The minimum power of the generated acoustic waves is selected in such a way that:

the power of a single wave generated by one epicenter would be insufficient to cause mechanical trauma/destruction of the treated biological tissue;

the combined power of the interference of the waves generated by adjacent epicenters would be sufficient to cause trauma of the desired level to the treated biological tissue.

In some preferred embodiments of the present invention, the initial power of each single acoustic wave is selected in such a way that mechanically traumatized biological tissue areas are formed both in the interference zone and in the zone at least immediately surrounding the epicenter of said acoustic wave.

In preferred embodiments of the present invention, the level of mechanical trauma is selected from the range starting from the level causing destruction of the cell membrane integrity and ending with the level causing full destruction of the cells in the tissue to be rejuvenated. Consequently, the effect of the present invention can stimulate regeneration of biological tissue with the destruction of entire cells of said tissue or with no destruction. The regeneration/rejuvenating effect can be achieved even with partial trauma to the biological tissue cells.

Other preferred embodiments include those where the acoustic waves are generated as directional acoustic waves, which also contribute to the localization and optimization of microtrauma site formation.

The set objective is solved by the fact that the formation of the desired acoustic waves is due to the surface explosive evaporation of tissues caused by the absorption of powerful laser pulses. A necessary condition for the realization of the regime of explosive vaporization of tissue with the generation of acoustic waves is the effective absorption of laser radiation by tissue. Effective absorption of laser radiation can be achieved either by choice of wavelength appropriate to the high absorption coefficient of irradiated tissue or using an additional chromophore with high absorption coefficient at the selected wavelength applied to the irradiated surface. Above said required dimensions of common mode acoustic waves sources and their relative placement are provided with an appropriate redistribution of light radiation energy in the cross-sectional plane of the laser beam with the formation on the surface of said biological tissue periodic structure with maxima and minima of the light energy.

The aforementioned and other benefits and advantages of the claimed method for the rejuvenation of biological tissue and restoration of functional properties, characteristics, and structure thereof, - - - will be further disclosed in detail in the examples of some possible preferred but non-limiting embodiments with references to the accompanied drawings and figures.

### DRAWINGS

FIGS. 1A to 1C show a schematic of the formation of microtrauma areas in biological tissue cells in one (first) of the possible embodiments (low power acoustic waves), wherein FIG. 1A shows the power of each acoustic wave is selected in such a way that they are not sufficient for the mechanical destruction of any components of the treated biological tissue, and FIGS. 1B and 1C showing microtrauma areas occurring only in interference 5 with hatching.

FIGS. 2A to 2C show a schematic of the formation of microtrauma areas in biological tissue cells in another (second) possible embodiment (high power acoustic waves), wherein FIG. 2A shows the power of each acoustic wave selected in such a way that they create areas of mechanical destruction of the treated biological tissue near the corresponding epicenter, and FIGS. 2B and 2C show that at further distribution, the wavefronts of acoustic waves generated by adjacent epicenters contact, waves interfere and create microtrauma areas in interference zones.

FIGS. 3A to 3C show a schematic of the formation of microtrauma areas in biological tissue cells in yet another (third) possible embodiment (directional acoustic waves), wherein FIG. 3A show acoustic waves travelling from corresponding epicenters on the surface of biological tissue into the biological tissue in predetermined directions, FIG. 3B shows the beginning of microtrauma area formation in biological tissue cells, and FIG. 3C shows acoustic waves generated as directional acoustic waves in order to create microtrauma deep tissue.

### DESCRIPTION

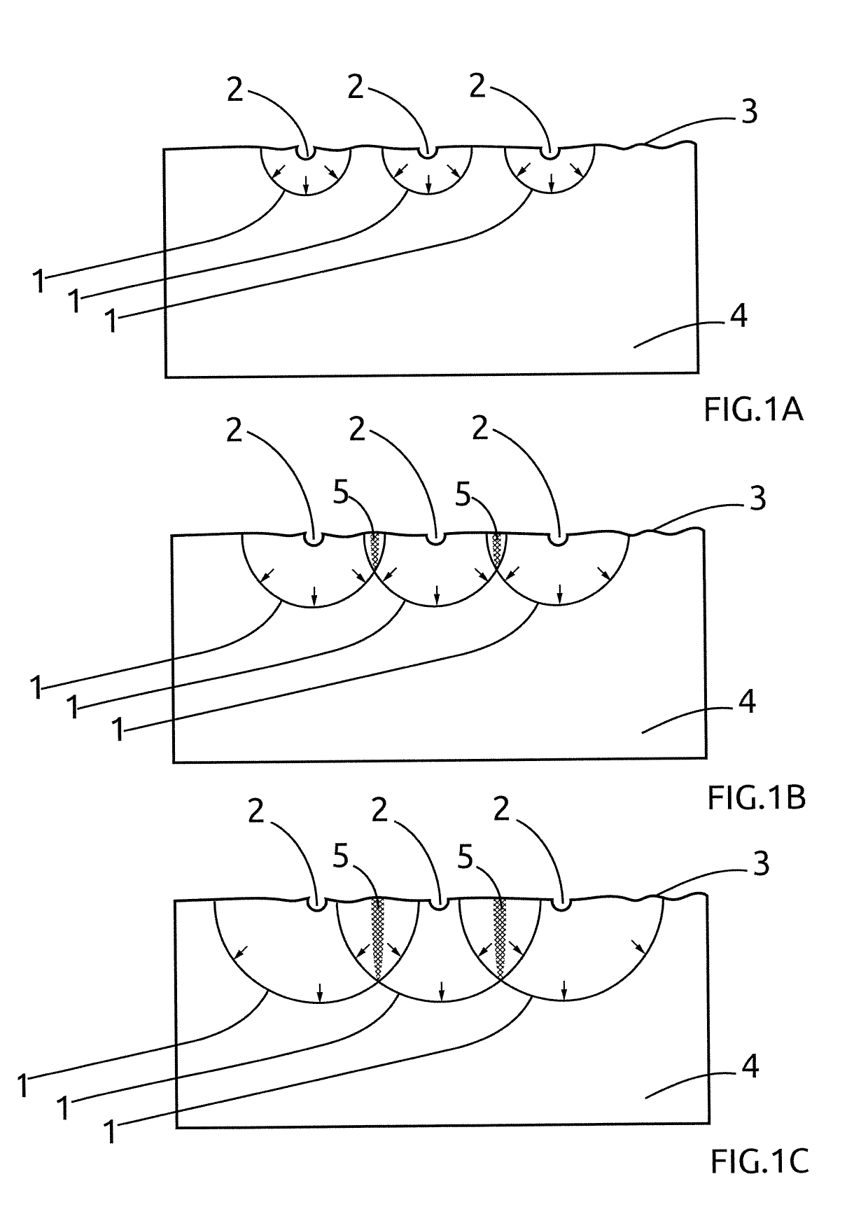

FIG. 1 shows step-by-step schematic of the formation of microtrauma areas in biological tissue cells in one of the possible embodiments, wherein acoustic waves 1 travel from corresponding epicenters 2 on surface 3 of biological tissue 4 into biological tissue 4, wherein the power of each acoustic wave 1 is selected in such a way that it is by itself not sufficient for the mechanical destruction of any components of the treated biological tissue 4 (FIG. 1A). Microtrauma areas will occur only in interference zones 5 (marked on FIGS. 1B and 1C with hatching) resulting at contact of wavefronts of acoustic waves 1 generated by adjacent epicenters 2.

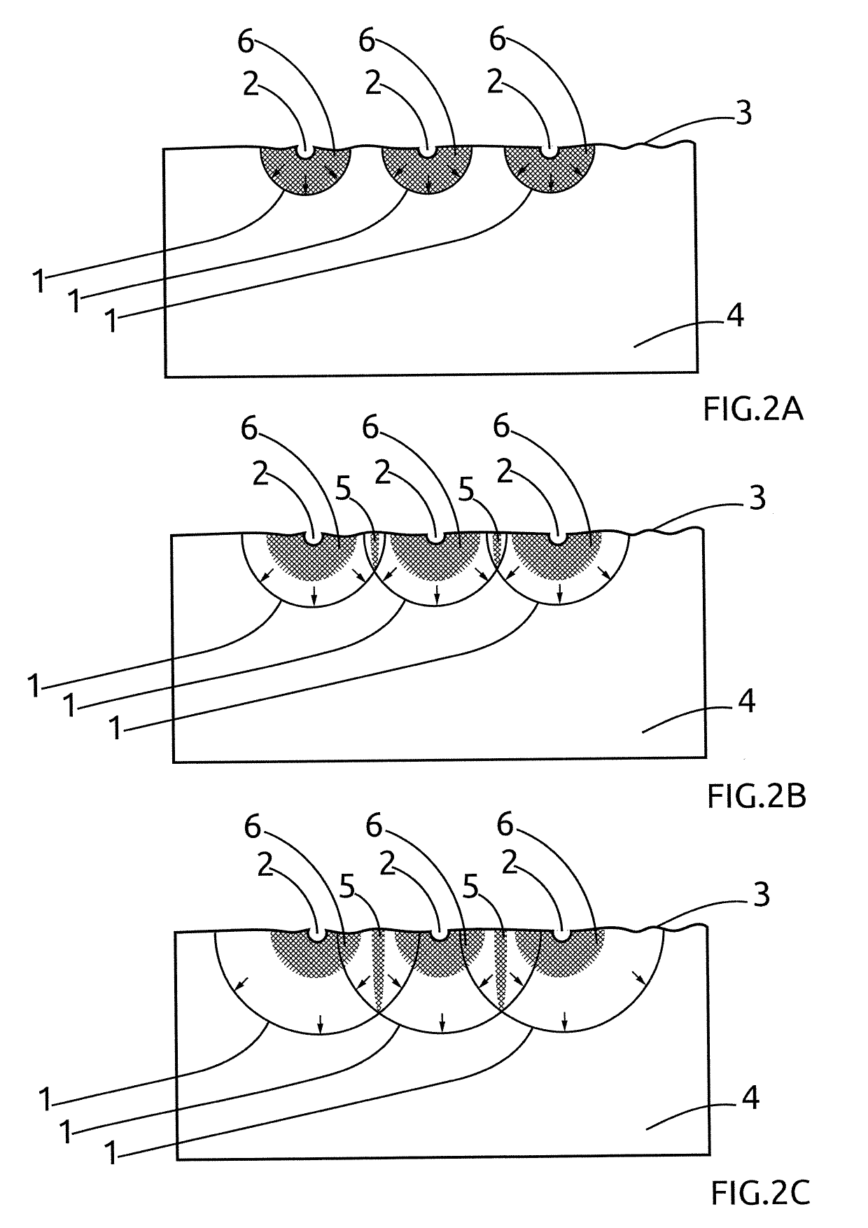

FIG. 2 shows step-by-step schematic of the formation of microtrauma areas in biological tissue cells in another (second) possible embodiment, wherein acoustic waves 1 travel from corresponding epicenters 2 on surface 3 of biological tissue 4 into biological tissue 4, wherein the power of each acoustic wave 1 is selected in such a way that it creates areas 6 of mechanical destruction of the treated biological tissue 4 near the corresponding epicenter 2 (FIG. 2A). At further distribution the wavefronts of acoustic waves 1 generated by adjacent epicenters 2 contact, waves interfere and create microtrauma areas (local trauma areas) in interference zones 5 (FIGS. 2B and 2C). Mechanical destruction zones 6 and interference zones 5 are marked with hatching on the drawings.

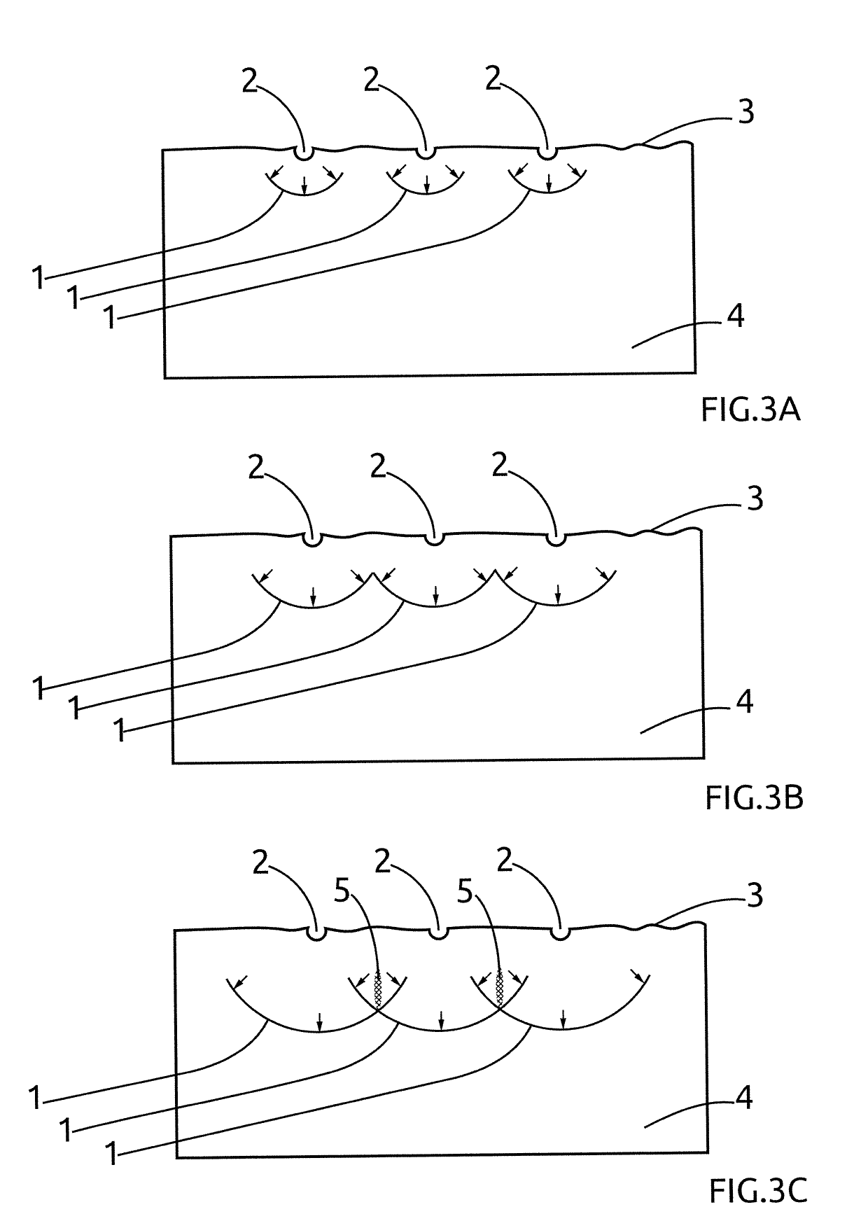

FIG. 3 shows step-by-step schematic of the formation of microtrauma areas in biological tissue cells in yet another (third) possible embodiment, wherein acoustic waves 1 travel from corresponding epicenters 2 on surface 3 of biological tissue 4 into biological tissue 4 in predetermined directions (FIG. 3A), wherein in order to create microtrauma areas in deep tissue (FIG. 3C), acoustic waves are generated as directional acoustic waves 1. FIG. 3B shows the beginning of microtrauma areas 5 formation in biological tissue cells 4. Microtrauma areas are formed in interference zones 5 of every two directional acoustic waves 1 generated by corresponding adjacent epicenters 2 (FIG. 3C). Interference zones 5 are marked with hatching on the drawings.

The claimed method is carried out as follows: Using any of the embodiments of the claimed method for the rejuvenation of biological tissue and restoration of functional properties, characteristics, and structure thereof, acoustic waves 1 are generated with the desired characteristics (power, frequency). Each acoustic wave 1 starts traveling into biological tissue 4 from corresponding epicenter 2. The microtrauma areas are created following the procedure of a particular embodiment of the claimed method.

For instance, for the embodiment of FIG. 1, since the minimum power of acoustic wave 1 is insufficient for the mechanical destruction of any of the components of treated biological tissue 4, the wave propagation is not accompanied by the destruction of biological tissue 4. However, interference with acoustic waves 1 propagated from corresponding adjacent epicenters 2 generates a localized cumulative power of acoustic wave 1 at the levels sufficient for the creation of a certain degree of mechanical trauma areas on biological tissue 4 in said interference areas 5. In addition, said areas will penetrate deeply inwards from surface 3 and will becomes zones of uneven (of the desired level) microtrauma of biological tissue cells 4.

Such tissue trauma is not intended to enlarge the contact area of living tissue with the aggressive environment, which minimizes the fibrous tissue formation. The power of acoustic waves 1 can be increased, according to the embodiment of FIG. 2, to reach the level when each separate acoustic wave 1 can independently destruct tissue. In that case, the effect will be produced in a somewhat different manner and will occur as follows: waves 1 propagating from each epicenter 2 into biological tissue 4 will trigger mechanical destruction thereof (areas 6 of mechanical destruction) until the power of said waves falls below the threshold. Any further propagation of waves 1 into biological tissue 4 is not accompanied by the destruction thereof, except interference zones 5 of waves 1 propagating from adjacent epicenters 2. Thus, as it is shown on FIGS. 2B and 2C, the trauma zone will include a completely destructed superficial tissue area (zone 6 of mechanical destruction) and local trauma zones (zones 5 of interference).

The observed visual effect in this case is the appearance of “frost”, i.e. a mechanically destructed area, on the treated surface. Additionally, varying the power of the acoustic wave can vary the depth of microtrauma area locations. Microtrauma to deep tissue 4 with subsequent regeneration thereof is conducted in accordance with the third embodiment (see FIG. 3) of the claimed method, i.e. by generating directional acoustic waves 1. In this case, the interference of acoustic waves 1 will take place only deep inside the biological tissue with no trauma to the superficial layers. Mechanical trauma areas will occur in interference zones 5.

Tissue regeneration in the embodiment of the present invention occurs faster compared to the prototype, because even in the effect of the second embodiment (see FIG. 2), the only fully mechanically destructed tissue is superficial tissue (area 6 of mechanical destruction), while deep tissue is not fully destructed but only traumatized (interference zones 5). In all other embodiments, including those not individually disclosed in the present specification, there is no full destruction of any biological tissue whatsoever.

The above description, illustrated with some possible non-limiting embodiments, therefore, demonstrates that although methods and corresponding devices for the regeneration/rejuvenation of biological tissue are known in medical practice, the claimed method and device provide novel and unexpected technical results. Said results are achieved primarily because for the rejuvenation of various biological tissue located at different depths, the areas of micotrauma can be created with no thermal effect applied, i.e., no evaporation or coagulation of any of the overlying tissue. Thus, tissue regeneration occurs with no fibrous cell growth, suggesting that not only visual but also actual rejuvenation takes place.

### REFERENCES

1. B. Eremeev, K. Kalaydzhyan—Lasers Against Wrinkles. Electronic almanac “Cosmetics & Medicine”. [Electronic resource]—Apr. 6, 2012. Access mode: http://daniel.ru/cm/arc/r403.htm.
2. Palomar-Rejuvenation. Website of the medical center RODEN. [Electronic resource]—May 4, 2012. Access mode:
   http://wwwxoden.by/cosmetology/palomar—omolojenie/
3. Application US2011/0218464 Al, publ. 09.08.201 1.
4. Application US2012/0016239 Al, publ. 01.19.2012.
5. Application US2012/0053458 Al, publ. 03.0112012.
   Patent JS6,997,923 B2, publ. 10.31.2002.

What is claimed:

1. A method for rejuvenation of biological tissue and restoration of functional properties, characteristics, and structure thereof by creating microtrauma sites in desired areas of biological tissue, followed by natural regeneration of corresponding biological tissue in specified areas, wherein said tissue are subjected to specified mechanically-induced trauma by affecting the desired areas with at least one site of interference of acoustic waves of an explosive nature generated by at least two sources and propagating through the tissue to be regenerated.

2. The method according to claim 1, wherein all epicenters of the acoustic waves are located at equal distances from one another, selected from a 10 μm to 1 cm range.

3. The method according to claim 2, wherein the level of mechanically-induced trauma is selected from the range between a level providing destruction of cell membrane integrity only and a level providing full destruction of cells in the tissue to be rejuvenated.

4. The method according to claim 3, comprising using a laser light source and a system for converting spatial distribution of beam intensity to form on the surface to be rejuvenated or the overlaying biological tissue a periodic structure with maxima and minima of light energy, forming, due to the effective absorption of energy in said maxima, a plurality of acoustic wave epicenters.
